(19) United States
(12) Patent Application Publication
(10) Pub. No.: US 2004/0223977 A1
(43) Pub. Date: Nov. 11, 2004

(54) Fusion peptide HIV vaccines

(75) Inventor: Don Diamond, Glendora, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 10/769,991

(22) Filed: Feb. 3, 2004

Related U.S. Application Data
(60) Provisional application No. 60/444,175, filed on Feb. 3, 2003.

(51) Int. Cl. A61K 39/21

(52) U.S. Cl. 424/188.1

(57) ABSTRACT

This invention provides a synthetic peptide vaccine against HIV and AIDS comprised of a potent helper epitope covalently linked to an HIV minimal CTL epitope. The vaccine induces potent epitope-specific CTL responses following a single administration in aqueous solution. This response can be further boosted with repeated administrations.

This application claims benefit of prior copending provisional application Ser. No. 60/444,175, filed Feb. 3, 2003, the disclosures of which are hereby incorporated by reference.

### STATEMENT OF FEDERALLY FUNDED RESEARCH

This invention was made with government support in the form of grant no. R21-AI44313 from the United States Department of Health and Human Services, National Institutes of Health, Division of AIDS. The United States government may have certain rights in the invention.

### BACKGROUND OF THE INVENTION

1. Field of the Invention

This invention pertains to peptide vaccine strategies for use against HIV and AIDS.

2. Description of the Background Art

The World Health Organization has estimated that since the start of the global pandemic, HIV has infected nearly 58 million people worldwide and that AIDS has claimed the lives of almost 22 million men, women and children. AIDS associated mortality has been greatly reduced by use of highly active anti-retroviral therapy (HAART) in Western countries, however the widespread use of these combination drug therapies is not possible for economic reasons in developing countries. Almost 50% of currently infected individuals are located in Southeast Asia and 40% in sub-Saharan Africa. Thus, the unavailability of effective anti-retroviral therapies for the majority of HIV-infected individuals is a major hurdle. In addition, both the emergence of drug-resistant HIV strains which likely will reduce the effectiveness of drug therapies and potential safety concerns regarding the long-term administration of potent anti-retroviral drugs makes the development of a safe, efficacious and globally available HIV vaccine all the more urgent. Moreover, prevention of HIV infection and HIV disease is far preferable to treatment. Therefore, a non-toxic and effective vaccine against HIV and AIDS would be highly desirable. However, no such vaccine is currently approved as safe and effective by the U.S. Food and Drug Administration.

An HIV vaccine which can stimulate CTL that recognize and kill HIV infected cells can be of great benefit to persons already infected with the virus as well as for prophylaxis. Cell-mediated immunity is generally considered to play the dominant role in restricting HIV replication during both the acute and chronic stages of infection. During the progression of HIV disease, HIV-specific CTL responses decay as the virus infects and destroys the activated CD4+ T helper cells required to coordinate and maintain the immune response. Early treatment with (HAART) during acute HIV infection results in a dramatic and prolonged suppression of HIV replication which can preserve CD4+ T cell function. However, when HAART is administered in the chronic stage of HIV infection, deterioration of HIV-specific CTL activity occurs due to a lack of HIV antigenic stimulation. Transient interruptions of HAART have been shown to preserve the integrity of HIV-specific CD4+ and CD8+ responses by providing the antigenic stimulation required to re-prime or boost the antiviral response. However, repeated interruptions in antiviral chemotherapy are undesirable as they increase the risk of generating drug-resistant viral strains within the infected host. Therefore, an effective CTL immunity-inducing vaccine would be an important part of treatment for HIV positive individuals in both chronic and acute stages of infection.

Potent cytotoxic T lymphocyte (CTL) responses are considered critical in the immunological defense against HIV, including resistance to infection, and long-term non-progression to AIDS in infected persons. Researchers have began to develop strategies to induce potent antiviral CTL responses using defined HLA-restricted minimal CTL epitopes from several HIV gene products. One approach is the administration of a DNA plasmid encoding several minimal epitopes of HIV linked together under the control of a constitutive promoter, however, it remains to be seen if DNA vaccination can be effective for human immunization.

Alternatively, CTL epitopes can be delivered as part of a synthetic peptide construct which can be engineered to include several restricted HLA haplotypes. According to conventional wisdom in the art, however, such peptide antigens often are non-immunogenic due to a number of intrinsic factors including incorrect processing by the immune system, rapid clearance, dilution effect, and lack of T cell help. For these types of vaccines to produce a useful, strong CTL response, the addition of powerful immunological adjuvants or chemical modification of the vaccine peptide is necessary. See, for example, Gahéry-Ségard, J. Virol. 74:1694-17031, 2000; Pialoux, AIDS 15:1239-1249, 2001; Belyakov et al., Nat. Med. 7(12):1320-1326, 2001; Belyakov et al., Proc. Natl. Acad. Sci. USA 95(4):1709-1714, 1998; Shirai et al., J. Immunol. 152(2):549-556, 1994. Unfortunately, many of the known and frequently used adjuvants fail to induce antigen-specific CTLs and many have associated side effects which make them unsuitable for human use.

Recently, bacterial and synthetic DNA have attracted attention as a safe and effective adjuvant for human use with the capacity to promote potent CTL responses following either parenteral or mucosal administration. Adjuvant activity has been associated with palindromic DNA sequences which contain unmethylated CpG groups flanked by two 5′ purine residues and two 3′ pyrimidines sometimes termed CpG DNA (optimally 5′ GpA-CpG-TpC or TpT′ 3′). CpG DNA directly stimulates antigen presenting cells (APCs) to produce cytokines (including TNF-α, IL-1, IL-6, IL-10, IL-12, GM-CSF) and to upregulate expression of MHC and crucial costimulatory molecules. CpG DNA may also act on B lymphocytes, inducing their proliferation and production of IL-6 and IL-10 and enhancing cytotoxicity and IFN-γ secretion by NK cells.

Previous attempts have been made to use HIV HLA-restricted synthetic CTL epitopes in the construction of a vaccine, however, these vaccines were found to function poorly as immunogens when formulated with Incomplete Freund's Adjuvant (IFA). T cell helper activity was lacking, therefore the CTL response could not be maintained. Exogenous T helper activity can be provided in trans by co-administering the pan HLA DR-binding epitope, PADRE. The PADRE sequence is a chemically defined and easily synthesized promiscuous T helper peptide epitope capable of binding with high affinity to a broad range of the most common HLA-DR types. Vaccines using this strategy, however, still are known to require formulation with a potent adjuvant to evoke a cytolytic response. Use of oligodeoxynucleotides (ODN) for clinical trial has proved safe and effective. Previously, peptide administered with ODN has been shown to stimulate CTL in a TH-independent manner, but only after repeated doses.

Current strategies, while promising, have not yielded any vaccines which induce a strong and durable CTL response to HIV which could be useful in public health, either to prevent HIV infection or to promote immune attack against HIV in infected persons. Therefore, new methods of prodrug HIV vaccines and HIV vaccine formulations are needed in the art.

### SUMMARY OF THE INVENTION

Accordingly, the present invention provides an HIV vaccine comprising a fusion peptide of an HIV CTL epitope and a T helper epitope, and optionally a DNA adjuvant. The CTL epitope may be HIV Pol₄₆₄₋₄₇₂ (SEQ ID. NO:1) or HIV gag₇₇₋₈₅ (SEQ ID NO:10). In another embodiment, the invention provides a method of vaccinating a patient in need thereof against HIV comprising administering a vaccine as described to the patient. In yet another embodiment, the invention provides a method of altering the immune response to HIV in a patient in need thereof comprising administering a vaccine as described to the patient. In yet a further embodiment, the invention provides a method of detecting HIV antigen-specific CD8⁺ T cells in a population of cells comprising contacting said cells with a tetramer reagent which comprises HIV Pol₄₆₄₋₄₇₂ (SEQ ID NO:1) or HIV gag₇₇₋₈₅ (SEQ ID NO:10).

### BRIEF DESCRIPTION OF THE DRAWINGS

FIG. 1 is a bar graph showing percent specific lysis of IV9 peptide loaded T2 cells at Effector: Target ratios of 25:1(black bar), 6:1(white bar) and 3:1(shaded bar).

FIG. 2 is a bar graph showing stimulation index of stimulated compared to unstimulated splenocytes as determined by thymidine incorporation at the indicated ODN concentrations.

FIG. 3 is a bar graph showing percent specific lysis of IV9 peptide loaded T2 cells at Effector: Target ratios of 25:1(black bar), 6:1(white bar) and 3:1(shaded bar).

FIG. 4 shows specific lysis results for splenocytes from animals immunized with different quantities of PADRE-IV9 fusion peptide with or without DNA adjuvant at the indicated Effector:Target ratios.

FIG. 5 shows specific lysis results for splenocytes from animals immunized with PADRE-IV9 fusion peptide with the indicated amount of DNA adjuvant at the indicated Effector:Target ratios.

FIG. 6 is a bar graph showing % specific lysis of target cells by cells immunized with fusion peptide alone or with DNA adjuvant by the intranasal or intrarectal routes.

FIG. 7 provides IV9-cell staining FACS results for unstained cells, IV9 tetramer-stained cells, and NV9 tetramer-stained cells. A-C show results using cells immunized with peptide alone; D-F show results using cells immunized with peptide and DNA adjuvant.

FIG. 8 provides data for % specific lysis of relevant and irrelevant peptide-loaded cells by effector cells from animals immunized with PADRE-IV9 fusion peptide alone (A) or with CpG DNA (B).

FIG. 9 provides cytotoxicity assay results for killing by immune spleen cells of Jurkat HLA A*0201 cells expressing full length HIV Pol, unmodified or ubiquitinated. See example 11.

FIG. 10 provides the same results for killing of T2 target cells that present an HIV Pol peptide or an irrelevant, control peptide.

### DETAILED DESCRIPTION OF THE PREFERRED EMBODIMENTS

The transgenic mouse model is widely accepted and routinely used by those of skill in the art to predict successful results in humans regarding the efficacy of peptide-based vaccines and HLA restricted CTL responses in humans. The transgenic mouse is the primary experimental animal model for investigation of the human immune system and this model system is well recognized in the art as correlating with results in humans and other mammals. See Ishioka et al., J. Immunol. 162: 3915-3925 (1999).

In these studies, transgenic HLA A2/K^{b} mice expressing the chimeric class I major histocompatability complex (MHC-I) HLA A2/K^{b} were used to investigate immunogenicity. The minimal HLA A*0201 restricted HIV CTL epitope derived from the HIV protein Pol was studied in conjunction with PADRE, both separately as individual epitopes and as a fusion peptide construct. Mice were immunized with a fusion peptide consisting of the HLA A*0201-restricted HIV CTL epitope Pol₄₆₄₋₄₇₂ (ILKEPVHGV; SEQ ID NO:1) covalently linked to the COOH terminus of the promiscuous T helper epitope PADRE, in the presence and absence of a CpG DNA adjuvant. No additional amino acid residues were included in the fusion peptide sequence to separate the two linked epitopes, since the PADRE molecule contains three alanine residues at the carboxyl-terminus which can serve as a natural spacing motif.

A single parenteral injection of 100 nmoles of the fusion peptide in normal (0.9%) saline stimulated a vigorous CTL response in HLA A2/K^{b} mice without the need for extraneous adjuvant. The potency of this aqueous fusion peptide vaccine was dramatically increased by including as little as 1 μg of phosphorothioated CpG DNA in the vaccine formulation. Using an HLA A2/K^{b} tetramer staining reagent, increased vaccine peptide-specific binding within the CD8+ population was shown to correlate with elevated cytolytic activity. These results demonstrate that the vaccines described here are effective in augmenting the immune response to HIV.

Synthetic or natural CTL epitopes such as SEQ ID NO:1 or SEQ ID NO:10 may be used to detect populations of T cells (CD8+) which recognize that epitope using tetrameric reagents according to methods known in the art. See, for example, Altman et al., Science 274:94-96, 1996 and U.S. Pat. No. 5,734,023, the disclosures of which are hereby incorporated by reference. Such methods are useful, for example, to diagnose HIV exposure or disease and to monitor the reactivity of T cells to HIV in infected persons.

A synergistic effect of surprising degree also was observed when both fusion peptide and CpG DNA were co-administered, whether the vaccine formulation was delivered parenterally, intranasally or intrarectally. The method of vaccinating against HIV with synthetic vaccine peptide thus provides potent cytolytic activity even in the absence of a traditional adjuvant. Fusion peptides constructed using restricted epitopes from a different HLA background (HLAA*1101) also were highly immunogenic in the correct genetic background (data not shown). By promoting potent cytolytic responses using HIV epitopes formulated with PADRE in conjunction with CpG DNA, HIV-specific immunity can be augmented or maintained during the chronic phase of HIV infection without the need to halt antiviral treatment.

Vaccines according to the present invention may be formulated according to any suitable method known in the art, and may contain any suitable and compatible diluent, carrier, preservative, pharmaceutical excipient or other ingredient. Suitable pharmaceutical carriers for administration intravenously, intraperitoneally, intramuscularly, transmucosally (for example intranasally., rectally, vaginally, buccally), transdermally, subcutaneously or any other route of administration are well known in the art and are contemplated for use with the invention.

Dosages which are suitable for any individual patient or vaccination subject are routinely determined by practitioners of skill in the art. Generally, a single dose of peptide of about 0.01 to about 10 mg/kg or preferably about 0.1 to about 1 mg/kg are useful. The vaccine may be administered one time, or booster vaccinations, for example a second administration, may be given. In general, 1-5 administrations or preferably 1-3 administrations or 1-2 administrations are given.

A DNA adjuvant may be included in the vaccine formulation. Generally, about 0.1 μg/kg. to about 2 mg/kg or preferably about 1 μg/kg to about 200 μg/kg per dose is sufficient to enhance the immune response to the peptide vaccine material. Synthetic oligonucleotide adjuvants may be used according to methods described in Jones et al., Vaccine 17(23-24): 3065-3071, 1999, the disclosures of which are hereby incorporated by reference. Persons of skill in the art of vaccination consider it routine to formulate vaccines with suitable doses of adjuvant.

Because both synthetic peptides and CpG DNA have been approved as safe for administration to humans, this vaccination strategy for the induction of potent CTL responses in human subjects is considered both safe and effective. Vaccines and methods of vaccination as described and claimed in this application may be used for both prophylaxis and treatment of HIV infection and HIV disease. Therefore, the vaccines may be administered to subjects who are either HIV-negative or HIV-positive, i.e. those seropositive or seronegative for anti-HIV antibodies or those testing positive or negative for presence of HIV. The inventive methods and compositions also may be used for parenteral or mucosal vaccination against infectious diseases caused by viral, bacterial and parasitic organisms and cancers. In addition, formulation of fusion peptides with alternative helper epitopes derived specifically from the pathogen or tumor of interest can boost pathogen- or tumor-specific T cell help. Examples of helper epitopes that also may be used include, for example, tetanus-specific peptides such as tetanus peptides 830-843, 590-603, 615-629, 639-652 and 947-967 (see, for example BenMohamed et al., Hum. Immunol. 61:764-779, 2000). In general, any helper T lymphocyte epitope known in the art may be used.

### EXAMPLES

### Example 1

### Production of CTL Epitope Vaccine Peptides

The HLA A*0201-restricted nonamer CTL epitopes HIV Pol₄₆₄₋₄₇₂ and human cytomegalovirus tegument protein pp65₄₉₅₋₅₀₃ and the promiscuous T helper epitope PADRE were synthesized individually or as PADRE-CTL epitope fusion peptides using standard Fmoc procedures which are known in the art. See Table 1 below (x=cyclohexylalanine). Molecular masses were confirmed by MALDI-TOF using a Kompact Probe™ (Kratos Analytical) instrument and all peptides were determined to be at least 80% pure by HPLC analysis.

TABLE I

Synthetic Minimal CTL and
Helper Epitope Peptides.

| | | | | |
|---|---|---|---|---|
| | | MW | | SEQ ID |
| Peptide | Name | (kDa) | Sequence | NO: |

| | | | | |
|---|---|---|---|---|
| Pan DR binding | PADRE | 1.354 | AKXVAAWTLKAAA | 4 |
| epitope | | | | |
| HIV Pol₄₆₄₋₄₇₂ | IV9 | 0.991 | ILKEPVHGV | 1 |
| PADRE-Pol₄₆₄₋₄₇₂ | PADRE-IV9 | 2.327 | AKXVAAWTLKAAAILKEPVHGV | 3 |
| fusion | | | | |
| HCMV PP65₄₉₅₋₅₀₃ | NV9 | 0.943 | NLVPMVATV | 2 |
| PADRE- | PADRE-NV9 | 2.582 | KSSAKXVAAWTLKAAANLVPMVATV | 13 |
| HCMVpp65₄₉₅₋₅₀₃ | | | | |
| HIV Gag₇₇₋₈₅ | SL9 | 0.981 | SLYNTVATL | 10 |
| PADRE-Gag₇₇₋₈₅ | PADRE-SL9 | 2.619 | KSSAKXVAAWTLKAAASLYNTVATL | 11 |
| Fusion | | | | |
| Human p53₁₄₉₋₁₅₇ | SV9 | 0.911 | STPPPGTRV | 12 |
| HBV | FV10 | 1.156 | FLPSDFFPSV | 7 |
| nucleocapsid | | | | |
| protein | | | | |

Synthetic ODN 1826 (5′ TCCATGACGTTCCTGACGTT 3′; SEQ ID NO:5), ODN 1984 (5′ TCCAGGACTTCTCTCAGGTT 3′; SEQ ID NO:6) and synthetic ODN 2006 (5′ TCGTCGTTTGTCGTTTTGTCGTT 3′; SEQ ID NO: 14) were synthesized with nuclease-resistant phosphorothioate backbones using methods known in the art (Alpha DNA; Montreal, Québec, Canada). See Moldoveanu et al., Vaccine 16(11-12):1216-1224, 1998, the disclosures of which are hereby incorporated by reference. The Na⁺ salts of the nucleic acids were resuspended at 5 mg/ml in 10 mM Tris (pH 7.0) with 1 mM EDTA and stored as 50 μl aliquots at −20° C. before dilution in normal saline prior to injection.

### Example 2

### Lymphocyte Proliferation Assays

A naïve transgenic A2/K^{b} spleen cell suspension was prepared as described for immunized splenocytes in Example 4. Following the removal of erythrocytes using red cell lysis buffer (0.15 M NH₄Cl, 1 mM KHCO₃, 0.1 mM Na₂EDTA in distilled H₂O) and subsequent washing, the concentration was adjusted to 4×10⁶ cells/ml in complete in vitro stimulation (IVS) medium. One hundred microliters of splenocyte suspension was plated onto 96 well U-bottomed plate along with 100 μl of serially diluted CpG DNA in serum-free IVS medium. Concanavalin A (Sigma, St. Louis, Mo.) at a concentration of 5 μg/ml and unstimulated splenocytes were used as positive and negative controls respectively. Following incubation in a humidified 5% CO₂ incubator for 48 hours at 37° C., 1 μCi of ³H-thymidine (TdR) was added to each well and plates were further incubated overnight as before. Cells were lysed by freeze/thaw and subsequently harvested onto a glass fiber filter using a cell harvester (Skatron Instruments, Norway). Levels of TdR uptake were determined by liquid scintillation using a TriLux counter (Wallac, Norway). Results were expressed as a stimulation index (SI) compared to unstimulated splenocytes.

### Example 3

### Cytotoxicity Assays

Cytolytic activity was determined using a 4 hour chromium release assay (CRA). T2 cells (ATCC) or Jurkat T cells transfected with the HLA A*0201 gene or JA2/R7Hyg T cells (Tsomides et al., J. Exp. Med. 180(4):1283-1293, 1994) in log growth phase, were used as targets. T2 target cells were loaded with 50 nmoles (1 ml) of relevant or non-relevant (control) peptide and 200 μCi of Na^{+ 51}CrO₄⁻ (ICN, Costa Mesa Calif.) in serum free T2 medium (RPMI supplemented with 10 mM HEPES, 2 mM L-glutamine, 100 μg/ml streptomycin and 100 U/ml penicillin) for 1 hour. Jurkat/HLA A2.1 target cells were infected with vaccinia viruses containing or expressing HIV pol gene (AA₅₈₇₋₁₄₃₅). All target cells, including JA2/R7Hyg cells (which are HIV-infected and do not need to be pulsed with peptides or infected with other viruses) were incubated with murine splenocytes as follows. After washing 3 times in complete IVS medium, targets were added to the effector splenocytes in triplicate at various effector: target ratios in a 96 well, round-bottom microplate in a final volume of 200 μl. Following incubation in a humidified 5% CO₂ incubator for 4 hours at 37° C., supernatants were harvested using a SKATRON® microplate harvesting system (Skatron Instruments, Norway) and analyzed for gamma emission. Percent specific lysis was calculated using the following formula: % specific lysis=[(test cpm-spontaneous cpm)/(total cpm-spontaneous cpm)]×100.

### Example 4

### Construction of a Ubiquitinated HIVpol Gene for Detecting CTL Activity Against Full-Length RT

The human ubiquitin gene, followed by the arginine codon and a lysine-rich (e^{k}) sequence (Suzuki and Vaishavsky, EMBO J. 18:6017-6026, 1999; Tobery and Sikiciano, J. Exp. Med. 185:909-920, 1997) was amplified using the following primer pair: primer A (sense; 5′-cttaagcttggtgcggccgccatgcagatctt-3′; SEQ ID NO:15) and primer B (antisense; 5′-taatactgacgctcgagcgggccctcgggaaac; SEQ ID NO:16). The PCR conditions were 94° C., 2 minutes; 25 cycles of 94° C., 30 seconds, 65° C., 30 seconds; 72° C., 40 seconds and then 72° C. for 1 minute. The resulting 363 bp Ub-R-e^{k} PCR product was gel-purified and cloned into the pSC11 insertion plasmid (modified with a polylinker) using Not1 and Apa1 restriction enzyme sites to generate Ub-R-e^{k}-pSC11. Chakrabarti et al., Mol. Cell. Biol. 5:3403-3409, 1985.

The HIV-1 pol gene, containing both RT and Integrase (IN) protein domains, was extracted and amplified from pNL4-3 (a plasmid containing a full-length clone of clade B HIV-1; Adachi et al., J. Virol. 59:284-291, 1986) using the following primer pair: primer C (sense; 5′-ttgatcgggcccattagccctattgagactgtacca‘3’; SEQ ID NO:17) and primer D (antisense; gaaggcctctaatcctcatcctgtctacttgccac; SEQ ID NO:18). The PCR conditions were 94° C., 2 minutes; 25 cycles of 94° C., 30 seconds; 62.2° C., 30 seconds; 72° C., 4 minutes; and then 72° C. for 10 minutes. The 2544 bp PCR product was gel-purified and cloned into Ub-R-e^{k}-pSC11 using Apa1 and Stu1 restriction sites to produce Ub-R-e^{k}-RT-In-pS11 (Ub-R-pol). This construct was verified by restriction enzyme digestion and DNA sequencing. The Ub-R-pol recombinant vaccinia virus (Ub-R-pol-VV) was generated by transfecting the Ub-R-pol-pSC11 plasmid into wild type VV-infected Hu TK⁻ cells as described by Diamond et al., Blood 90:1751-1767, 1997. Ub-R-pol-VV was subjected to 4 rounds of simultaneous screening and selection using color reaction of substrates (Bluogal™, Sigma-Aldrich) to β-galactosidase and resistance of BrdU. The expression of the ubiquitin-modified and unmodified RT-In expressed from VCF21 was detected by western blot using MAb21 (data not shown). Ferris et al., Virology 175:456-464, 1990. The unmodified HIV-1 clade B pol-expressing VV (vCF21) was expanded from seed stock. See Flexner et al., Virology 166:339-349, 1998.

### Example 5

### HIV-Specific CTL Response After Single Immunization With IV9 Peptide and PADRE

All immunization experiments were carried out using 6 to 12 week old transgenic HLA A*0201/K^{b} (hereafter A2/K^{b}) mice bred onto the C57BL/6 background. See BenMohamed et al., Hum. Immunol. 61:764-779, 2000; the disclosures of which are hereby incorporated by reference. For parenteral administration, animals received peptide vaccine as a single subcutaneous injection at the base of the tail in a final volume of 100 μl saline. For intranasal administration, peptide solution was applied as droplets over the external nares of awake animals in a final volume of 30 μl saline. Rectal immunization was conducted by depositing the vaccine solutions into the colon of awake animals via the anus using a Gilson P200 pipette and sterile yellow tip in a final volume of 15 μl. A total of 3 mice were immunized per group unless otherwise stated.

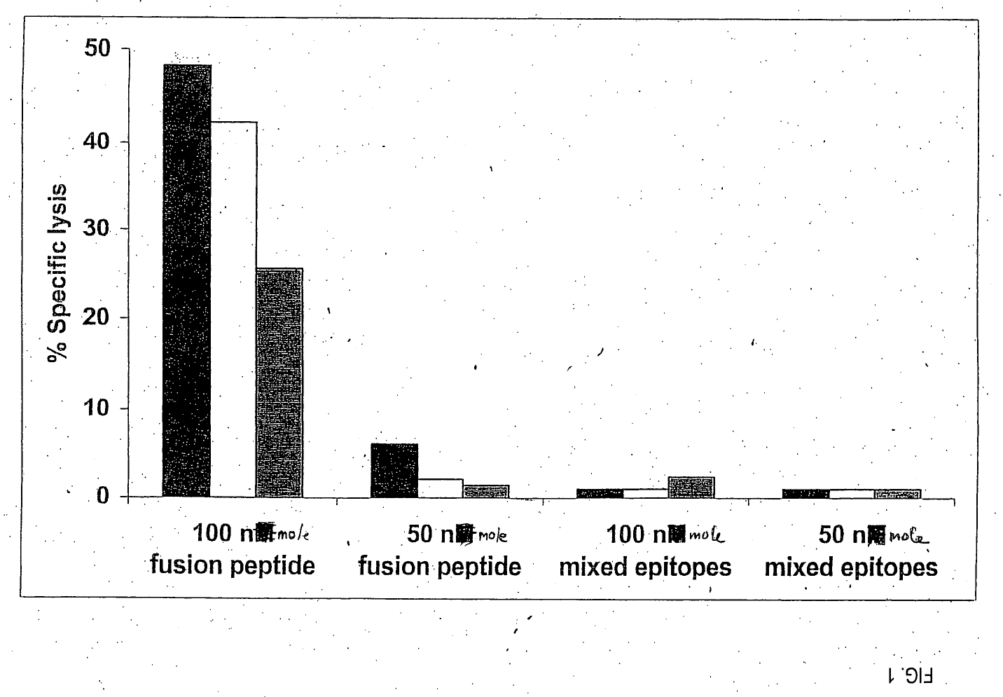

Spleens then were retrieved from sacrificed animals 14 days after vaccination and a single cell splenocyte suspension prepared by passing the cells through a 70 μm Falcon cell strainer (Becton Dickinson Labware, Franklin Lakes, N.J.) using the plunger from a sterile 1 ml syringe. Splenocytes were subjected to 1 round of in vitro stimulation (IVS) as follows and then subjected to assay. Syngeneic naïve splenocytes were cultured in complete IVS medium (RPMI supplemented with 10% fetal calf serum, 10 mM HEPES, 50 μM 2-mercaptoethanol, 2 mM L-glutamine, 100 μg/ml streptomycin and 100 U/ml penicillin) for 3 days in the presence of 2.5 μg/ml lipopolysaccharide (LPS) (Sigma) and 7 μg/ml dextran sulphate. These LPS blasts were then pulsed with the relevant peptide in serum-free IVS medium at 200 nmoles per ml and subsequently irradiated (3000 rads). Splenocytes from the immunized animals were co-cultured with the indicated peptide-loaded LPS blasts in complete IVS medium at a ratio of 3:1 for 7 days, with the addition of 10% rat T-Stim™ (Collaborative Biomedical Products, Bedford Mass.) on day 3 of stimulation. CTL responses were quantitated by chromium release assay at effector: target (E:T) ratios of 25:1 (black bar), 6:1 (white bar) and 3:1 (shaded bar) an expressed as % specific lysis. See FIG. 1.

When HLA A2/K^{b} mice were immunized with 100 nmoles of the minimal CTL epitope co-administered with an equal amount of PADRE (PADRE+IV9) emulsified in IFA, a weak but discernible peptide-specific CTL response was observed following a single round of in vitro stimulation. This observed cytolytic activity could be further enhanced by subjecting CTLs to additional rounds of IVS. As expected, the administration of 100 nmoles PADRE+IV9 formulated in saline failed to prime the animals for an HIV-specific CTL response (see FIG. 1, mixed epitopes).

### Example 6

### HIV-Specific CTL Response of the Single Immunization with IV9-PADRE Fusion Peptide

Immunization with a fusion peptide consisting of the IV9 sequence covalently attached to the COOH terminus of the PADRE sequence (SEQ ID NO:3) in saline was performed as in Example 4. The magnitude of the CTL response in A2/K^{b} mice inoculated with either the PADRE-IV9 fusion peptide or mixed PADRE+IV9 minimal epitopes prepared in saline (or emulsified in IFA; data not shown) was assessed by chromium release assay after a single round of IVS using relevant IV9 peptide-loaded T2 cells at E:T ratios of 25:1 (black bar), 6:1 (white bar) and 3:1 (shaded bar). See FIG. 1. Levels of non specific cytotoxicity, determined against T2 cells loaded with an irrelevant HBV nucleocapsid protein peptide FV10 (FLPSDFFPSV; SEQ ID NO:7) at the same E:T ratios were subtracted from the results shown.

When formulated with IFA, CTL responses were observed in mice immunized with either the fusion peptide vaccine or the unfused minimal epitopes as previously noted (data not shown). Interestingly, a dose-dependent potent peptide-specific CTL response (approximately 50% specific lysis at an E:T of 25:1) was observed in animals receiving 100 nmoles of PADRE-IV9 fusion peptide administered in saline as shown in FIG. 1. The level of lytic activity was diminished to approximately 10% specific lysis at an E:T of 25:1 when the dose of fusion peptide was reduced to 50 nmoles. No cytolytic responses were observed when mice were administered the mixed unfused peptides (PADRE+IV9 epitopes) in saline at either the 100 nmole or 50 nmole dose.

### Example 7

### TH-CTL Fusion Peptide HIV Immunogenicity Augmentation

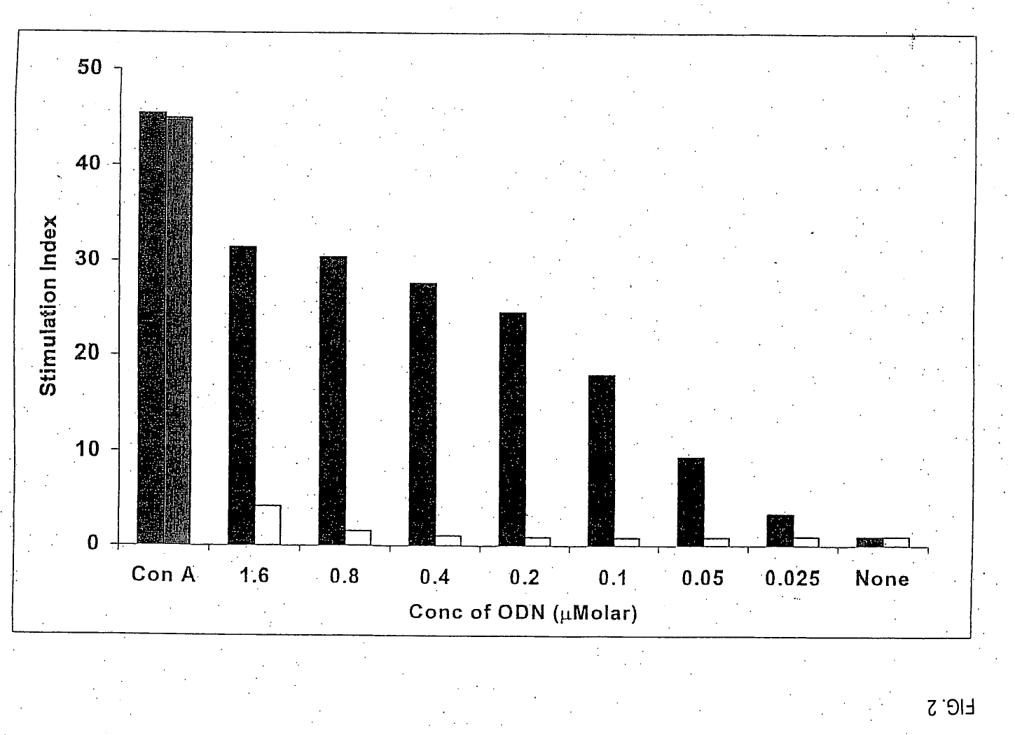

In vitro analysis of the proliferative effect of two synthetic ODNs was conducted using naïve HLA A2/K^{b} splenocytes. Each nucleic acid was manufactured with a nuclease-resistant phosphorothioate backbone. Proliferative responses were determined by ³H-thymidine incorporation using naïve transgenic A2/K^{b} splenocytes cultured with immunostimulatory ODN 1826 (SEQ ID NO:5; black bar) or control ODN sequence 1984 (SEQ ID NO:6; white bar) for 48 hours. Results are presented as stimulation index (SI) compared to unstimulated splenocytes. See FIG. 2. Concanavalin A treated splenocytes were included as a positive control.

The results demonstrate that ODN 1826, which contains two copies of the immunostimulatory GACGTT motif, caused a potent generalized proliferative response when added to naïve HLA A2/K^{b} splenocytes, even at low molar concentrations. However, the control sequence (ODN 1984; SEQ ID NO:6), which contains the same number of individual nucleotide residues randomized to eliminate the CpG motifs, produced little or no proliferation even at the highest concentration tested (1.6 μM).

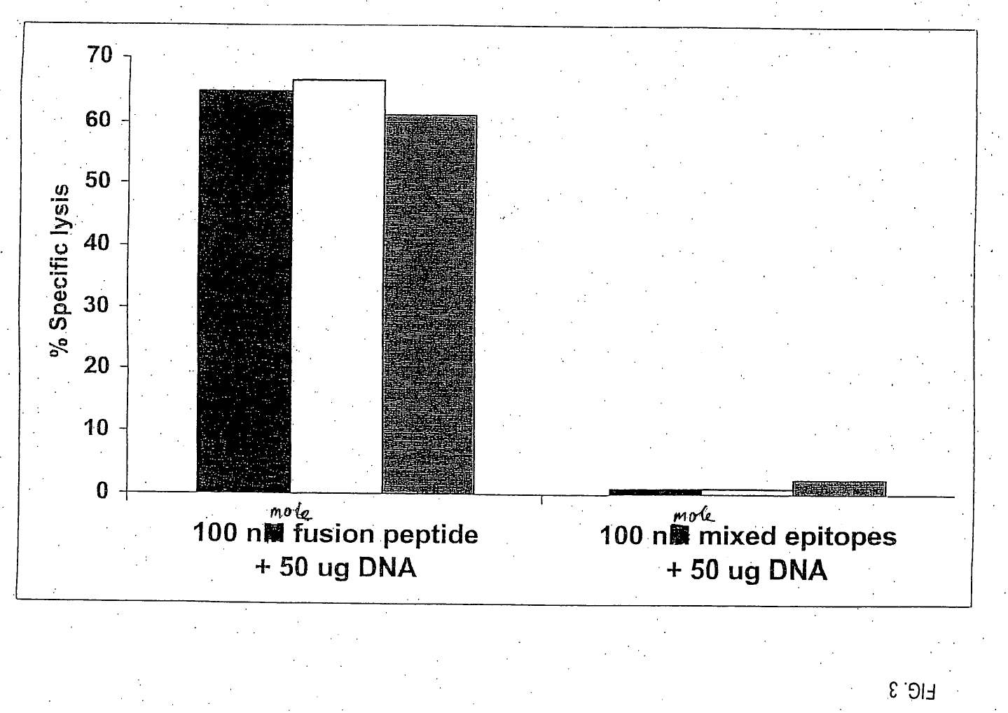

Based on this observation, ODN 1826 was formulated with the PADRE-IV9 fusion peptide in aqueous solution to assess whether the epitope-specific CTL responses following parenteral vaccination could be further increased. A2/K^{b} mice were immunized once subcutaneously with 50 μg ODN 1826 and either 100 nmoles PADRE-IV9 fusion peptide or 100 nmoles of unfused minimal epitope plus PADRE prepared in saline. Fourteen days later, splenocytes from immunized mice were stimulated in vitro for 7 days with irradiated IV9-loaded syngeneic LPS blasts. Cytolytic T cell responses were determined by ⁵¹Cr-release assay using relevant IV9 peptide-loaded T2 cells at E:T ratios of 25:1 (black bar), 6:1 (white bar) and 3:1 (shaded bar). See FIG. 3. Levels of nonspecific cytotoxicity determined against irrelevant FV10 (SEQ ID NO:7) loaded T2 cells at the same E:T ratios were subtracted from the results shown.

Administration of 100 nmoles PADRE-IV9 fusion peptide in conjunction with 50 μg ODN 1826 resulted in extremely high levels of peptide-specific cytotoxicity at all E:T ratios tested. These responses ranged from 1.32-fold to 2.2-fold higher than the cytolytic responses observed at the same E:T ratios when the fusion peptide was administered in the absence of CpG DNA (compare FIG. 1). The formulation of ODN 1826 with the unfused minimal T_{H} and CTL epitopes failed to induce any evidence of a CTL response against the minimal IV9 epitope under these experimental conditions (FIG. 3).

### Example 8

### Dose-Dependent CTL Responses against Fusion Peptide/CpG DNA Formulation

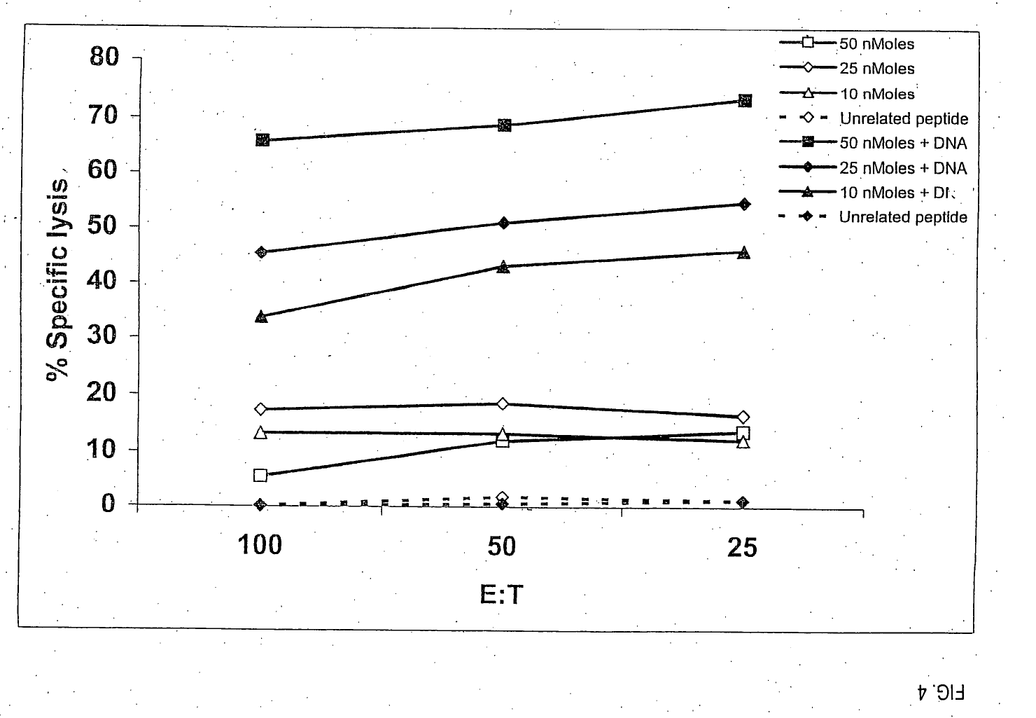

To optimize the formulation of fusion peptide and CpG DNA required to stimulate a maximal CTL response, a dose titration study was performed in two stages. Initially, animals were immunized once subcutaneously with varying quantities of PADRE-IV9 fusion peptide, ranging from 10 nmoles to 50 nmoles, either alone or in combination with 25 μg of ODN 1826, in saline. Spleens were retrieved 14 days after immunization and CTL activity determined following a single round of in vitro stimulation with irradiated IV9-loaded syngeneic LPS blasts. Cytolytic T cell responses were determined by ⁵¹Cr-release assay using relevant and irrelevant peptide-loaded T2 cells as targets at the indicated E:T ratios indicated in FIG. 4.

As illustrated in FIG. 4, minimal CTL responses were observed when fusion peptide was given in the absence of DNA (open symbols). However, addition of 25 μg ODN 1826 to the vaccine formulation markedly enhanced CTL responses to PADRE-IV9 fusion peptide at immunogen doses as low as 10 nmoles (FIG. 4). The observed synergistic effect of the immunostimulatory DNA appeared to be directly related to the quantity of fusion peptide in the vaccine formulation and was more pronounced at higher doses. For example, between 10-15 nmoles of peptide at an E:T ratio of 25, the resultant cytotoxicity was approximately 15%. There was an approximate 3-fold, 3.5-fold, or 5-fold stimulation in the presence of 10 nmoles, 25 nmoles, or 50 nmoles CpG DNA respectively. No cytolytic activity was noted in any group when target cells were loaded with NV9, an irrelevant control HLA A*0201-restricted CTL epitope from HCMV (SEQ ID NO:2).

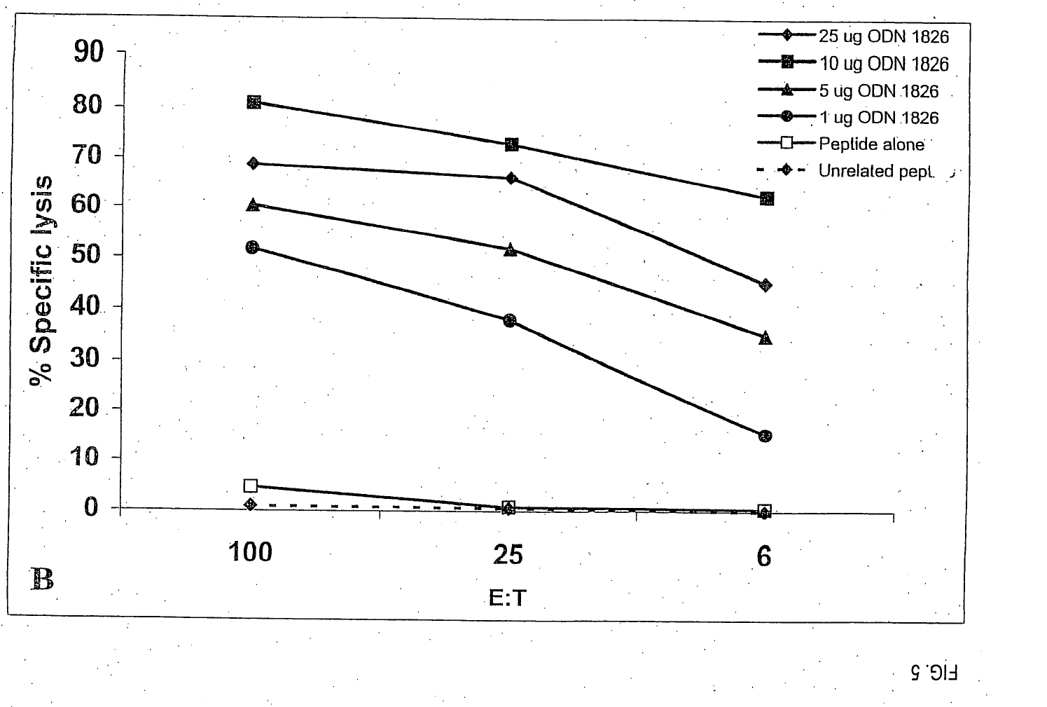

To determine the minimal amount of CpG DNA required to augment the CTL response, groups of transgenic A2/K^{b} mice were immunized with 50 nmoles PADRE-IV9 alone or together with varying amounts ODN 1826 ranging from 1 μg to 25 μg, in saline. Transgenic A2/K^{b} mice were immunized subcutaneously on day 0. Following the administration of 50 nmoles PADRE-IV9 fusion peptide, CpG DNA is capable of augmenting the immune response with as little as 1 μg ODN 1826 per dose (FIG. 5). CTL responses plateau between 10 μg and 25 μg of ODN 1826 at an E:T of 25 and higher. At an E:T of 6, there was no clear plateau even at 25 μg CpG DNA, although the theoretical maximum of 100% lysis was close to being achieved.

### Example 9

### Fusion Peptide Vaccination Formulation via the Mucosal Route

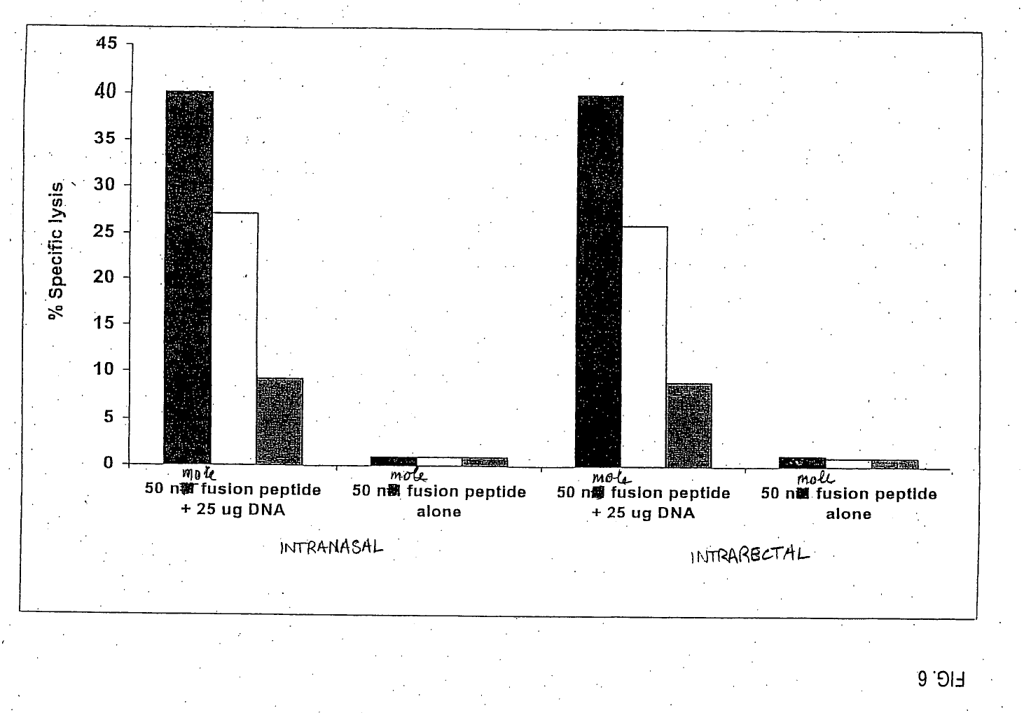

The major portals of entry for a large number of infectious agents are the mucosal surfaces of the genitourinary, gastrointestinal and respiratory tracts. The possibility that mucosal immunization, which has the potential to induce both systemic and mucosal immune responses, would be effective against such mucosal pathogens, including HIV, was investigated. To evaluate the ability of the fusion peptide vaccine to induce a systemic CTL response following mucosal delivery, A2/K^{b} mice were administered 50 nmoles of fusion peptide vaccine with or without 25 μg ODN 1826 via the intranasal or intrarectal route. Following 7 days in vitro stimulation, lytic activity of splenocytes was determined by ⁵¹Cr-release assay using relevant IV9 peptide-loaded T2 cells at E:T ratios of 50:1 (black bar), 12:1 (white bar) and 3:1 (shaded bar). See FIG. 6. Levels of nonspecific cytotoxicity determined against irrelevant FV10 loaded T2 cells at the same E:T ratios were subtracted from the results shown. Potent CTL activity was observed following immunization with fusion peptide and ODN 1826 via both routes whereas no lytic activity was observed in the absence of CpG DNA.

In the absence of CpG DNA, administration of a single dose of fusion peptide at either mucosal site failed to induce peptide-specific cytolysis following in vitro culture of splenocytes with syngeneic IV9-loaded LPS blasts for 1 week. In contrast, the addition of 25 μg of CpG DNA to the vaccine formulation markedly enhanced the resultant CTL activity in splenocyte cultures for both routes of delivery. In the presence of CpG DNA, cytotoxicity was increased approximately 50-fold compared to the response obtained against fusion peptide alone at an E:T ratio of 100 after a single administration. Furthermore, the levels of lytic activity were comparable following either intranasal or intrarectal immunization of the fusion peptide with CpG DNA.

### Example 10

### Detection of HIV-Reactive CTL

To precisely quantitate antigen-specific CD8+ T cells within the lymphocyte population, chimeric HLA A2/K^{b} tetramer reagents were constructed according to known methods containing either the IV9 or irrelevant (control) NV9 minimal CTL epitope as follows. Sequences corresponding to the extracellular domain of the HLA A2/K^{b} heavy chain were amplified by PCR from cDNA prepared from A2/K^{b}-expressing murine splenocytes by reverse transcription of total cellular mRNA. Primers HLA35 (5′-cgcgcgaattcaggaggaatttaaaatgggctcccactccatgagg-3′; SEQ ID NO: 8) and HLA683 (5′-gcgcaagcttttaacgatgattccacaccattttct gtgcatccagaatatgatgcagggatcccggctcccatctcagggtga; SEQ ID NO:9) were used with the following PCR conditions: preliminary 15 minutes at 95° C. to activate the HotStart™ Taq polymerase, followed by 30 cycles of 30 seconds at 94° C., 30 seconds at 60° C., 1 minute at 72° C., and a final extension step for 8 minutes at 72° C. The amplicons were cloned into the pHN1 prokaryotic expression system using restriction enzyme sites within the PCR oligonucleotide primers. PHN1 is an E. coli expression plasmid vector with a tac promoter and the β-lactamase ampicillin resistance gene. See Garboczi et al., Proc. Natl. Acad. Sci. USA 89:3429-3433, 1992. The host strain was XA90F′ lacI⁹. A Bir A biotinylation substrate site was engineered at the COOH terminus of the HLA A2/K^{b} heavy chain by incorporation of the sequence into one of the PCR primers. HLA A2/K^{b} tetramers were produced using a minor modification of the procedure employed by the NIAID Tetramer Core Facility. HLA A2/K^{b} heavy chain and β₂-microglobulin (β₂-M) were produced from Escherichia coli XA90 following transformation with the pHN1 constructs. Recombinant heavy chain and β₂-M were refolded in the presence of either the IV9 or NV9 peptide (SEQ ID NOS:1 or 2, respectively). Refolded monomeric complexes were then concentrated, biotinylated using the enzyme BirA (Avidity Inc., Denver, Colo.) and subsequently purified by FPLC chromatography using a Sephacryl S300 gel filtration column, followed by a MonoQ ion exchange column. Tetrameric complexes were formed by conjugation with streptavidin-PE (Pharmingen).

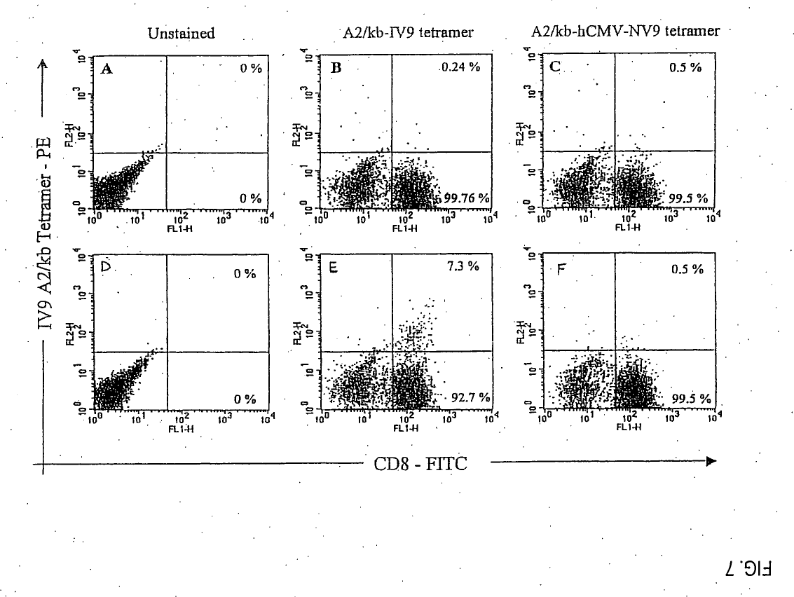

For flow cytometric analysis, Anti murine CD8 conjugated to FITC was purchased from Becton Dickinson (San Jose, Calif.). Staining and washing of cells was performed using FACS buffer (PBS containing 0.5% FCS, and 0.1% sodium azide). For direct fluorescent labeling, one million cells were incubated with either A2/K^{b}/IV9 or A2/K^{b}/NV9 tetramer (1 μg per sample in 20 μl) for 30 minutes at 4° C. and washed with cold FACS buffer. Cells were then incubated with anti-CD8-FITC (2 μl per sample in 20 μl) at 4° C. for a further 30 minutes. After completing the staining process, cells were again washed, then analyzed immediately using a Becton-Dickinson FACSCalibur flow cytometer. Data were analyzed using CellQuest software for MacIntosh. See FIG. 7, in which panels A, B and C show results for cells derived from the spleens of mice immunized with PADRE-IV9 fusion peptide alone and panels D, E and F show results for cells derived from the spleens of mice immunized with PADRE-IV9 fusion peptide plus CpG DNA.

CTL obtained from transgenic A2/K^{b} mice immunized with 50 nM PADRE-IV9 fusion alone or 50 nmoles PADRE-IV9 fusion peptide plus 25 μg ODN 1826 were analyzed after a single round of in vitro stimulation. Cytometric analysis of unstained cells (FIGS. 7A and 7D) and cells stained with either anti CD8-FITC and A2/K^{b}-IV9 tetramer-PE (FIGS. 7B and 7E) or anti CD8-FITC and A2/K^{b}-NV9 tetramer-PE (FIGS. 7C and 7F) was performed after 7 days in culture. Plots shown were gated on small lymphocytes and the numbers represent the percentage of CD8 positive cells in the respective quadrants.

In vitro cultures derived from transgenic mice immunized with 50 nmoles PADRE-IV9 fusion peptide with 25 μg CpG DNA contained a subset of CD8+ T cells (7.3% of total CD8+ cells) which were capable of binding A2/K^{b}-IV9 tetramer (FIG. 7E). In contrast, only 0.24% of CD8+ cells obtained from a similar culture originating from animals administered 50 nmoles PADRE-IV9 fusion alone could bind the IV9 tetramer (FIG. 7B). A comparable background level of 0.5% was observed when each culture was stained with an A2/K^{b} tetramer containing the unrelated NV9 epitope from HCMV (see FIGS. 7C and 7F).

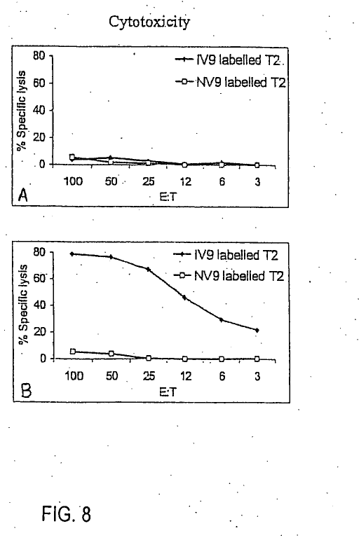

The ability of CD8+ cells to bind A2/K^{b}-IV9 tetramer was associated with potent lytic activity. Lytic activity of effector cells for each sorted group was determined by ⁵¹Cr-release assay against relevant IV9 and irrelevant NV9 peptide-loaded T2 target cells at the indicated E:T ratios. See FIGS. 8A (effectors immunized with PADRE-IV9 (SEQ ID NO:3)) and 8B (effectors immunized with SEQ ID NO:3) with CpG DNA). Effector CTL obtained from mice immunized with both fusion peptide and CpG DNA were very efficient killers, with approximately 80% lysis observed at an E:T ratio of 100 (FIG. 8B). No significant cytolytic activity was observed for cells derived from A2/K^{b} mice administered 50 nmoles of fusion peptide in the absence of DNA (FIG. 8A).

### Example 11

### Recognition of Endogenously Processed HIV-pol Expressed from Vaccinia Virus

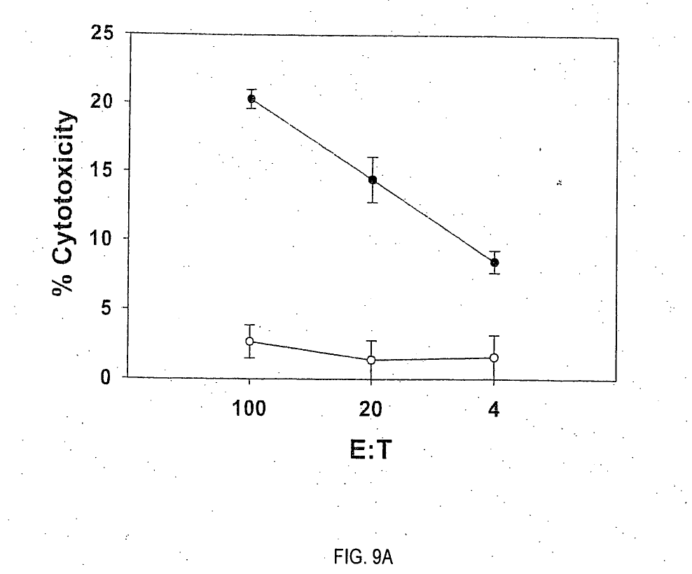

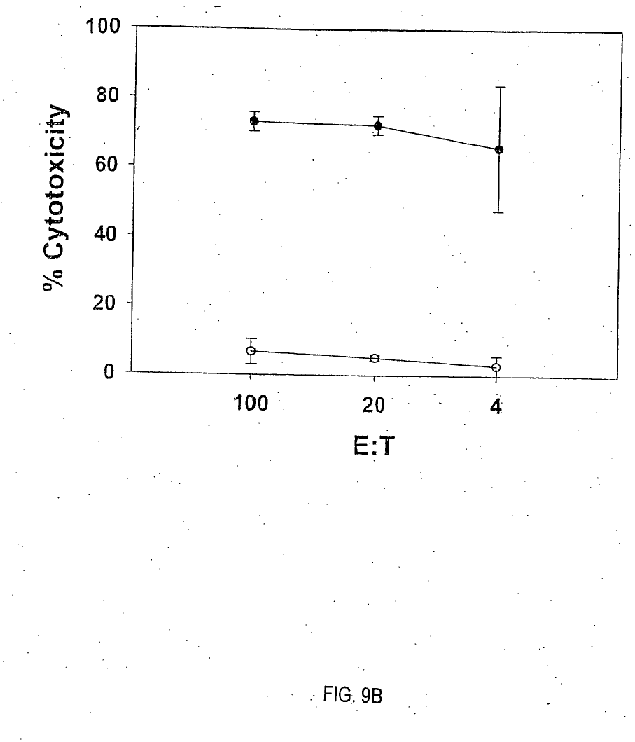

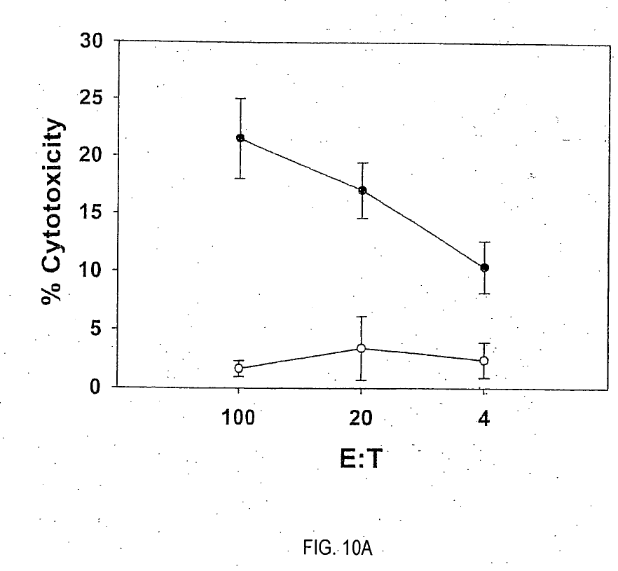

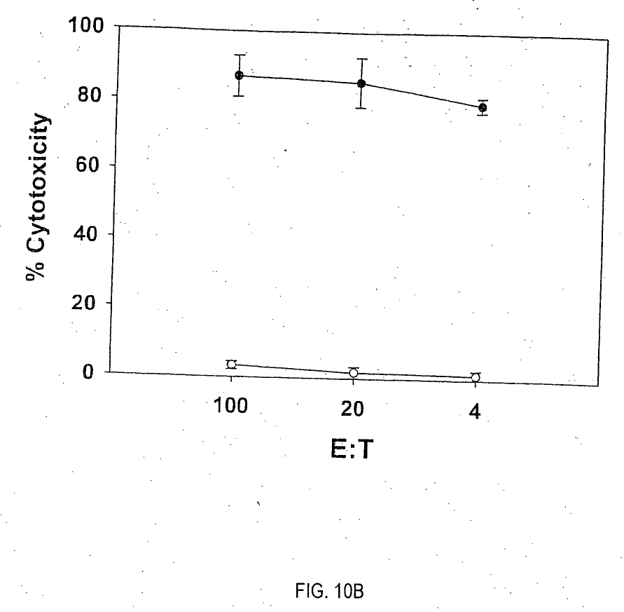

To detect recognition of processed full-length HIV-pol protein from vaccinia virus, A2/K^{b} mice were immunized subcutaneously at the base of the tail with 25 μg CpG DNA (#1826) and 100 nmole (FIGS. 9A and 9B) or 50 nmole (FIGS. 10A and 10B) SEQ ID NO:3. The immunogen consisted of HIV Pol₄₆₄₋₄₇₂ covalently attached to the COOH terminus of the PADRE sequence, in saline solution. Two weeks after immunization, the mice were sacrificed. Spleen cells were subjected to two rounds of in vitro stimulation with the peptide of SEQ ID NO:1 (HIV Pol₄₆₄₋₄₇₂). FIGS. 9A and 10A show results of cytotoxicity assays carried out using Jurkat target cells that express HLA A*0201 and that are infected with the reverse transcriptase gene product, either unmodified (HIV-pol VV; open circles) or modified by ubiquitination and substitution of the initial methionine with arginine (UbR-HIV-pol VV; closed circles). The effector: target (E:T) ratios were as given in FIGS. 9A and 10A.

### Example 12

### Recognition of HIV Pol₄₆₄₋₄₇₂ Peptide

Example 11 was repeated using T2 target cells that had been loaded with either the HIV Pol₄₆₄₋₄₇₂ peptide (SEQ ID NO:1) or the irrelevant peptide of SEQ ID NO:12. See FIG. 9B (100 nmole SEQ ID NO:3 immunogen) and FIG. 10B (50 nmole SEQ ID NO: 3 immunogen). Closed circles represents results with T2 cells loaded with SEQ ID NO:1 and open circles with SEQ ID NO:12.

### Example 13

### Recognition of HIV-Gag-specific Murine CTL After Immunization with SEQ ID NO:11

SEQ ID NO:11 (25 or 50 nmoles) was mixed with 25 μg, CpG DNA (ODN 1826) in 100 μl saline solution and administered subcutaneously at the base of the tail of HLA A2.1/Kb transgenic mice. After 12-14 days, mice were sacrificed and splenic effector cells were stimulated in culture either once or twice with peptide (SEQ ID NO:10). A chromium release assay was performed after a one-week in vitro stimulation using T2 target cells which had been previously pulsed with either SEQ ID NO:10 or SEQ ID NO:1. After subtracting non-specific recognition (SEQ ID NO:1), the specific recognition for mice immunized with 50 nmoles fusion peptide at different E:T ratios was as follows: E:T 100, 33%; E:T 20, 30%; E:T 4, 9.3%. The corresponding recognition for mice immunized with 25 nmoles fusion peptide were 49%, 62% and 41%, respectively.

### Example 14

### Recognition of Endogenously Processed HIV-Gag Expressed for Vaccinia Virus

Mice also were immunized with 25 nmoles, SEQ ID NO:11 for testing of recognition of full-length HIV gag protein in vaccinia virus ubiquitinated at the amino terminus. After two in vitro stimulations with SEQ ID NO: 10, the target cells (Jurkat cells expressing HLA A*0201) were lysed specifically at 40.3%, 30.7% and 12.3% at E:T ratios of 100, 20 and 4, respectively. Background recognition of vaccinia virus was minimal. The same experiment performed with mice immunized with 50 nmoles SEQ ID NO:11 resulted in specific lysis of 57%, 34.2% and 7.7% at the same E:T ratios. Again, background lysis was negligible.

1. An HIV vaccine which comprises a fusion peptide of an HIV CTL epitope and a T helper epitope.

2. An HIV vaccine of claim 1 wherein said HIV CTL epitope is selected from the group consisting of HIV Pol₄₆₄₋₄₇₂ (SEQ ID NO:1) and HIV gag₇₇₋₈₅ (SEQ ID NO:10).

3. An HIV vaccine of claim 1 wherein said T helper epitope is selected from the group consisting of PADRE and a tetanus peptide epitope.

4. An HIV vaccine of claim 1 which further comprises a DNA C adjuvant.

5. An HIV vaccine of claim 4, wherein said DNA adjuvant is a CpG DNA adjuvant.

6. A method of vaccinating a subject in need thereof against HIV, comprising administering to said subject an HIV vaccine according to claim 1.

7. A method of claim 6 wherein said subject is HIV-negative.

8. A method of claim 6 wherein said subject is HIV-positive.

9. A method of altering the immune response to HIV in a subject in need thereof, comprising administering to said subject an HIV vaccine according to claim 1.

10. A method of claim 9 wherein said subject is HIV-negative.

11. A method of claim 9 wherein said subject is HIV-positive.

12. A method according to claim 6 wherein said HIV vaccine is administered once.

13. A method according to claim 6 wherein said HIV vaccine is administered more than once.

14. A method according to claim 9 wherein said HIV vaccine is administered once.

15. A method according to claim 9 wherein said HIV vaccine is administered more than once.

16. A method of detecting HIV antigen-specific CD8⁺ T cells in a population of cells comprising contacting said cells with a reagent which comprises a peptide selected from the group consisting of HIV Pol₄₆₄₋₄₇₂ (SEQ ID NO:1) and HIV gag₇₇₋₈₅ (SEQ ID NO:10).


SEQUENCE LISTING (18 sequences)

SEQ ID NO: 1 - protein, 9 aa, Human immunodeficiency virus
ILKEPVHGV

SEQ ID NO: 2 - protein, 9 aa, Human cytomegalovirus
NLVPMVATV

SEQ ID NO: 3 - protein, 22 aa, Artificial
  PADRE - HIV Pol464-472 fusion peptide
AKXVAAWTLKAAAILKEPVHGV

SEQ ID NO: 4 - protein, 13 aa, Artificial
  Pan DR binding epitope from T helper cells
AKXVAAWTLKAAA

SEQ ID NO: 5 - DNA, 20 bp, Artificial
  oligonucleotide adjuvant
tccatgacgttcctgacgtt

SEQ ID NO: 6 - DNA, 20 bp, Artificial
  oligonucleotide adjuvant
tccaggacttctctcaggtt

SEQ ID NO: 7 - protein, 10 aa, Hepatitis B virus
FLPSDFFPSV

SEQ ID NO: 8 - DNA, 46 bp, Artificial
  oligonucleotide primer for HLA A2/Kb heavychain amplification
cgcgcgaattcaggaggaatttaaaatgggctcccactccatgagg

SEQ ID NO: 9 - DNA, 84 bp, Artificial
  oligonucleotide primer for HLA A2/Kb heavy chain amplification
gcgcaagcttttaacgatgattccacaccattttctgtgcatccagaatatgatgcagggatcccggctcccatctcagggtga

SEQ ID NO: 10 - protein, 9 aa, Human immunodeficiency virus
SLYNTVATL

SEQ ID NO: 11 - protein, 25 aa, Artificial
  PADRE - HIV Gag77-85 fusion peptide
KSSAKXVAAWTLKAAASLYNTVATL

SEQ ID NO: 12 - protein, 9 aa, Homo sapiens
STPPPGTRV

SEQ ID NO: 13 - protein, 25 aa, Artificial
  PADRE - HCMV pp65 495-503 fusion peptide
KSSAKXVAAWTLKAAANLVPMVATV

SEQ ID NO: 14 - DNA, 23 bp, Artificial
  oligonucleotide adjuvant
tcgtcgtttgtcgttttgtcgtt

SEQ ID NO: 15 - DNA, 32 bp, Artificial
  oligonucleotide primer for human ubiquitin gene amplification
cttaagcttggtgcggccgccatgcagatctt

SEQ ID NO: 16 - DNA, 33 bp, Artificial
  oligonucleotide primer for human ubiquitin gene amplification
taatactgacgctcgagcgggccctcgggaaac

SEQ ID NO: 17 - DNA, 36 bp, Artificial
  oligonucleotide primer for HIV-1 pol gene amplification
ttgatcgggcccattagccctattgagactgtacca

SEQ ID NO: 18 - DNA, 35 bp, Artificial
  oligonucleotide primer for HIV-1 pol gene amplification
gaaggcctctaatcctcatcctgtctacttgccac